(19) United States
(12) Patent Application Publication
(10) Pub. No.: US 2026/0357516 A1
(43) Pub. Date: Nov. 12, 2026

(54) CELLULOSE HYDROLYSIS APPARATUS FOR HIGH SOLIDS LOADING

(71) Applicant: EPISOME BIYOTEKNOLOJIK URUNLER SANAYI TICARET ANONIM SIRKETI, Gebze/Kocaeli (TR)

(72) Inventor: Murat BALABAN, Uskudar/Istanbul (TR)

(21) Appl. No.: 19/488,717

(22) PCT Filed: May 30, 2023
(86) PCT No.: PCT/TR2023/050489

§ 371 Date: Nov. 28, 2025

(51) Int. Cl. D21C 11/00; C12M 1/00; C12M 1/40

(52) U.S. Cl. CPC D21C 11/0007; C12M 21/18; C12M 45/06

(57) ABSTRACT

The present invention relates to an apparatus (1) for use in cellulose hydrolysis. The apparatus (1) comprises a channel (10) for guiding a stream of cellulose-containing waste in a flow direction (FD). The apparatus (1) further comprises one two or more shaft(s) (20) arranged to rotate around respective longitudinal axes (A), such that, when rotated, one or more side surfaces (71) of said shaft(s) (20) exhibit a linear velocity vector orthogonal to the axis (A). The two or more shafts (20) include one or more protrusions (70) that radially extend away from the axis (A). The present invention further proposes a method for cellulose hydrolysis.

### TECHNICAL FIELD

The present invention relates to a chemical engineering apparatus for use in enzymatic hydrolysis, second generation biofuel production and biorefining. In particular, the present invention relates to a cellulose hydrolysis apparatus capable to process high solids loading.

### BACKGROUND

Cellulose is a polysaccharide formed from glucose monomers. Polysaccharide molecules in pluralities form elongated fibres that constitute respective, macromolecular yarns. Cellulose molecules firmly bonded to each other form “crystalline cellulose”. On the other hand, if the cellulose molecules are loosely bonded to each other when compared to the crystalline cellulose, the respective structure is named as “amorphous cellulose”.

Water retention ability of amorphous cellulose is considerably higher when compared to that of the crystalline cellulose, because water molecules can easily enter in-between loosely bonded cellulose molecules (fibres) in a macromolecular yarn of amorphous cellulose. Furthermore, also enzymes can be easily introduced in-between such fibres of an amorphous cellulose yarn. Accordingly, the amorphous cellulose has a relatively low resistance against cellulase enzymes, when compared to that of crystalline cellulose.

Cellulose hydrolysis is used in second-generation bioethanol and biogas production processes. The cellulose hydrolysis can be chemical or enzymatic. Chemical hydrolysis employs acids such as sulphuric acid, whereas enzymatic hydrolysis makes use of various enzymes that degrade cellulose into its monomers.

Enzymatic hydrolysis of cellulose can be exemplified as follows: materials that contain cellulose fibres (e.g., straw), are subjected to chemical pre-treatment, and then (enzymatically) hydrolysed in an aqueous suspension in the presence of enzymes, e.g., for 24 to 72 hours at temperatures between 50 °C and 55 °C. This hydrolysis step is usually performed at a low extent of dry matter percentage, at which the suspension shows fluid behaviour. The suspension is subjected to mixing using mixers, and simultaneously, cellulose yarn particles that buoy in the suspension are separated therefrom. The cellulose molecules are hydrolysed into glucose, resulting in a molasses.

Paper sludge as a cellulosic waste is a non-fluid-like, highly viscous, material that includes cellulose fibres and yarns along with fillers. Paper sludge has a high extent of water retention capability. Due to its cellulose content, the paper sludge has an important potential for being used in biofuel production processes based on fermentation. Due to being an industrial waste that has to be disposed of, as a raw material, input cost attributed to paper sludge is considered to be zero.

Paper production process results in loose bonds between cellulose fibres; therefore, the cellulose yarns in paper sludge are exposed, allowing introduction of aqueous fluids in-between the fibres. Hence, paper sludge has a much higher extent of water retention ability when compared to other, ligneous or straw-based materials. This fact results in very high viscosities in aqueous suspensions of paper sludge, which precludes mixing in industrial conditions.

Paper sludge even shows solid-like behaviour when the dry matter content reaches or exceeds 20 wt. %. Due to difficulties in mixing, enzymatic hydrolysis of such solid-like paper sludge is extremely difficult.

More specifically;
Solids load ratio are low in hydrolysis that take place in a liquid medium. For instance, a 1000 kg of total medium weight can include 900 kg of an aqueous carrier (that can contain buffering components, surfactants etc.), 99 kg of cellulose, and 1 kg of enzymes. Such systems can be considered to be liquid.
Systems that have a higher solids load ratio can include 50 wt. % to 80 wt. % of water with regard to the total weight of a respective medium. Such systems employ much lower water content when compared to the above-mentioned liquid systems; and allow the use of volumetrically smaller reactors to hydrolyse the same amount of dry cellulose when compared to a respective liquid system.
In hydrolysis systems with low solids (or: dry) loadings, the water retention ability of delignified cellulosic material such as water sludge is much higher. Therefore, the viscosity is very high even with 1 wt. % of dry matter content with regard to the total weight of the respective medium. Such viscosity demands a high extent of energy consumption in mixing the medium. Hydrolysis with said low solids loading require high-volume mixers. Furthermore, the solid content has to be kept low in order to enable the use of industrial mixers. These measures dramatically increase the energy consumption per unit solid matter content in the medium.
In the case of low solids loading, mixers stir the water but the consumed mixer power cannot be directly and efficiently exerted onto the particles that are suspended in the respective medium. Thus, the torque and shear force of the mixer cannot be efficiently exerted onto the particles.
Hydrogen bridges between the water and cellulose molecules allow the capability of water retention. On the other hand, the macromolecular structure of cellulose yarns is constituted via the hydrogen bonds between cellulose molecules; and this fact results in a recalcitrance, that is, these hydrogen bonds between cellulose molecules can hinder the introduction of water and enzymes in-between cellulose fibres in a yarn.
In particular, due to the above-mentioned firm bonding between cellulose fibres, the interaction of crystalline cellulose with aqueous medium of a respective suspension occurs only in a superficial level. This results in a bulk movement of cellulose in the suspension, because the aqueous medium (or water itself) cannot penetrate through hydrogen bridges in-between cellulose fibres in crystalline cellulose yarns. Hence, enzymes in the aqueous medium cannot efficiently be introduced in-between said cellulose fibres, and the respective enzymatic reaction occurs mainly in a superficial level. To overcome this issue, the level of crystallinity in the cellulose can be decreased by various methods such as steam explosion, ammonia fiber expansion (AFEX) and diluted acid treatment. Such measures introduce an increased processing cost.

An advanced cellulose hydrolysis apparatus for use with high solids loading is described in WO 2023/063895 A1. The apparatus that is described in said document provides a high extent of grinding. Yet, it is desired to further enhance the extent of grinding of loadings with high solids content.

Accordingly, it is required to improve in processing equipment for use in cellulose hydrolysis.

### SUMMARY

The primary object of the present application is to overcome the above-mentioned shortcomings of the prior art. Another object of the present invention is to propose an apparatus and method that enable the minimization in the amount of enzymes necessary per cellulose unit for effectively hydrolysing the same. A further object of the present invention is to propose an apparatus and method that enable the processing of paper sludge with a relatively high solid load, thereby minimizing energy and apparatus investment costs. An even further object of the present invention is to propose an apparatus and method that enable an increased cellulose hydrolysis capacity, with minimized residence time in hydrolysis reaction.

The present invention achieves said objects with the features that constitute the appended independent claims.

The present improvement provides an apparatus and method that introduce an effective grinding of cellulose fibres and yarns. By such grinding, the crystallinity of cellulose can be decreased to a further extent when compared to the prior art. Hence, the water retention and enzymes receiving capability of cellulose is increased. Accordingly, the surface contact between enzymes and cellulose is maximized, thereby effectively maximizing the hydrolysis rate. This fact enables a minimized residence time and hydrolysis reactor size without compromising capacity, effectively hydrolysing high solid load cellulose wastes without increasing costs related to investment and operation. Hence, the following are achieved by the apparatus and method proposed herein:
the minimization in the amount of enzymes necessary per cellulose unit for effectively hydrolysing the same;
the processing of paper sludge with a relatively high solid load, thereby minimizing energy and apparatus investment costs; and
an increased cellulose hydrolysis capacity, with minimized residence time in hydrolysis reaction.

### BRIEF DESCRIPTION OF FIGURES

FIG. 1 is perspective view of an exemplary apparatus according to the present invention, showing a state where one or more shafts are at a first position with regard to a channel.

FIG. 2 is front view of the exemplary apparatus shown in FIG. 1, along the flow direction. The channel is omitted in FIG. 2 for emphasizing the shafts.

FIG. 3 shows a perspective view based on FIG. 2.

FIG. 4 shows a series of shafts arranged parallel to each other.

FIG. 5 shows a top perspective view based on FIG. 4.

FIG. 6 shows a side view of an exemplary shaft for an apparatus within the scope of the present application.

FIG. 7 shows a close-up view of the detail J from FIG. 6.

### DETAILED DESCRIPTION

With reference to the figures briefly described above, the present invention proposes an apparatus (1) for use in cellulose hydrolysis. The apparatus (1) comprises a channel (10) for guiding a stream of cellulose-containing waste in a flow direction (FD). The apparatus (1) further comprises two or more shaft(s) (20) each being arranged to rotate around respective axes (A).

The two or more shafts (20) respectively include one or more protrusions (70) that radially extend away from the axis (A). Thus, when rotated, one or more side surfaces (71) of said shaft(s) (20) exhibit a linear velocity vector around said axis (A). When compared to prior art apparatus with cylinders without protrusions, a comparable extent of linear velocity can be achieved on side surfaces (71) of protrusions (70) that are provided on a relatively low diameter shaft (20). In the case where the shafts (20) are made from a similar material, the weight of the shafts (20) are inevitably lower than the cylinders in the prior art apparatus.

Also, the protrusions (70) provide an increased extent of contact surfaces that contribute to grinding of the waste. Particularly when compared to the prior art apparatus with cylindrical side surfaces, at a section across the flow direction (FD), the length of side surfaces (71) in contact with the waste is increased to a great extent per flow area. Hence, the protrusions (70) provide the exertion of a much greater extent of shear onto the waste.

It can be thus considered that, when the shaft (20) is rotated at a rotational frequency, a side surface (71) on the protrusion (70) periodically approximates/approaches/reciprocates to a complementary surface on a protrusion (70) of an adjacent shaft (20). In other words; when the shaft(s) (20) are rotated, the side surfaces (71) and complementary surfaces cooperate in grinding the waste.

It can be further considered that such complementary surface can be a side surface (71) of a protrusion (70) on a further, adjacent shaft (20). Such shafts (20) that cooperate with each other can be arranged such that, the respective axes (A) of the couple of shafts (20) that are adjacent to each other are substantially parallel to each other.

With these features, a high extent of mechanical tension and pressure are simultaneously applied onto a respective cellulosic waste, to grind the same. Thus, any hydrogen bridges/bonds in-between cellulose yarns are easily loosened, and simultaneously, an enzyme-containing aqueous medium can be introduced into the cellulose. As a result, the proposed system (and the respective method) facilitates and expedites the cellulose hydrolysis reaction.

In a possible embodiment, the protrusions (70) can have side surfaces (71) that are provided with indentations. The indentations enhance the grinding by increasing the contact surface, and also contributes to mixing in the waste stream. Thus, the length of side surfaces (71) in contact with the waste is increased to an even greater extent per flow area. Hence, the protrusions (70) provide the exertion of an even greater extent of shear onto the waste. With this measure, the side surfaces (71) serve in provision of roughness, thereby further enhancing the grinding. The roughness enhances the extent of grinding; thus, increases the efficiency and rate of the cellulose hydrolysis.

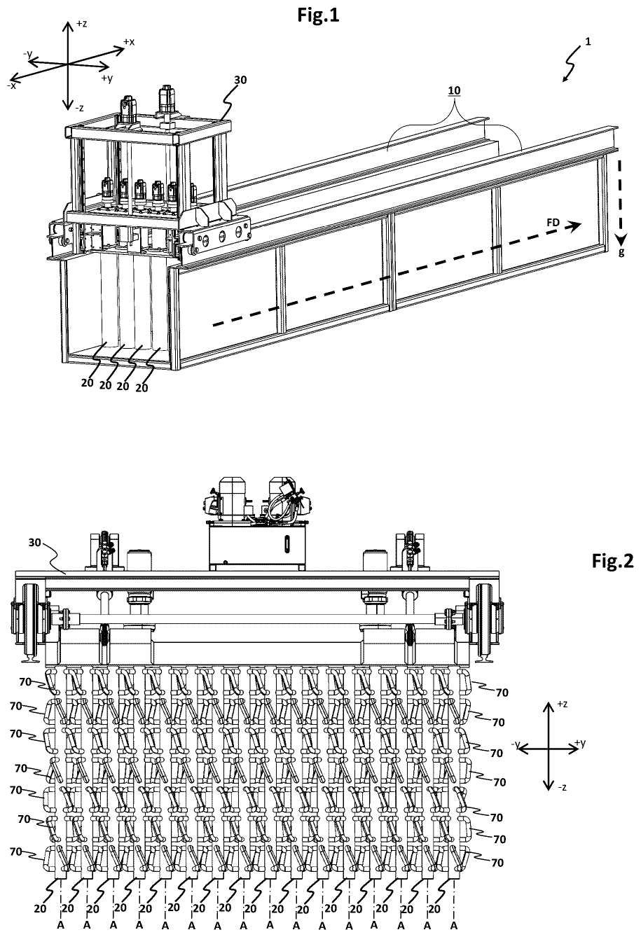

FIG. 1 shows perspective view of an exemplary apparatus (1) according to the present invention, showing a state where the two or more shafts (20) (here: a plurality thereof) are at a first position with regard to the channel (10). In FIG. 1, the shafts (20) are schematically represented without showing structural details such as the protrusions (70), merely for visualisation of an exemplary positioning thereof in the apparatus (1). Here, the shafts (20) are at a state where they are introduced into the channel (10), to contact and grind a waste stream that can be provided in the channel (10) when the apparatus (1) is in use. In other words, the shafts (20) are positioned (here: lowered) for contacting a waste stream flowing through the channel (10) along the flow direction (FD).

FIG. 2 is front view of the exemplary apparatus shown in FIG. 1, along the flow direction. Here, an exemplary plurality of protrusions (70), and their possible positioning on respective shafts (20) are visualised.

In accordance with the teaching above, the present invention further proposes a method for cellulose hydrolysis. The method can be performed for instance with the apparatus (1) described above. All of the technical effects and advantages of the possible features of the apparatus (1) that are described in the present specification are available with the method when a respective embodiment of the apparatus (1) is employed. Thus, the method can be considered as the use of a respective embodiment of the apparatus (1) according to the present application.

The proposed method includes the following actions:
guiding a stream of cellulose-containing waste in a flow direction (FD) along a channel (10); and
grinding the waste between respective protrusions (70) that radially extend away from respective axes (A) of two or more shafts (20) that rotate around said axes (A).

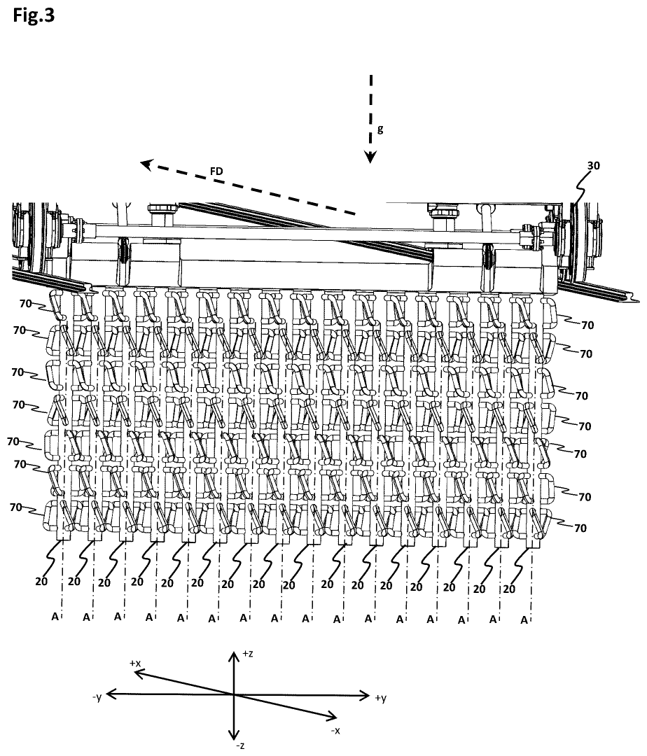

In a preferred embodiment of any of the above-discussed versions of the proposed apparatus (1), the two or more shaft(s) (20) can be arranged to have their respective axes (A) extending along the gravity vector (g) and orthogonal to the flow direction (FD) when the apparatus (1) is in use. FIG. 1 to FIG. 3 constitute examples to such embodiment. When the apparatus (1) is in use, the waste stream is horizontally guided along the flow direction (FD) that is substantially orthogonal to the axes (A); thus, the waste stream is effectively kneaded by the shafts (20).

In a preferred embodiment of any of the above-discussed versions of the proposed apparatus (1), the two or more shaft(s) (20) are each arranged to rotate in a respective rotational direction that is opposite to one or more adjacently arranged further shafts (20). Thus, cooperating respective side surfaces (71) of two adjacently arranged shafts (20) prompt the waste in a direction parallel to the flow direction (70). That is, the waste around a shaft (20) is prompted in the flow direction (FD) at a first lateral side of the shaft (20); whereas, at a second lateral side that is opposite to said first lateral side with regard to the axis (A), the waste is prompted opposite to the flow direction (FD). Thus, the mixing of the stream is promoted to increase the extent in processing of the waste.

In a preferred embodiment of any of the above-discussed versions of the proposed apparatus (1), said two or more shaft(s) (20) (e.g., each of them) are arranged to rotate such that respective side surfaces (71) on the protrusions (70) exhibit a linear velocity other than that of a corresponding complementary surface. In other words, the two or more shaft(s) (20) are each arranged to rotate such that respective side surfaces (71) on the protrusions (70) exhibit a relative linear velocity higher than zero with respect to complementary side surfaces (71) on protrusions (70) of adjacently arranged shaft(s) (20). This enhances the kneading that is exerted onto the waste stream. Thus, the grinding is enhanced.

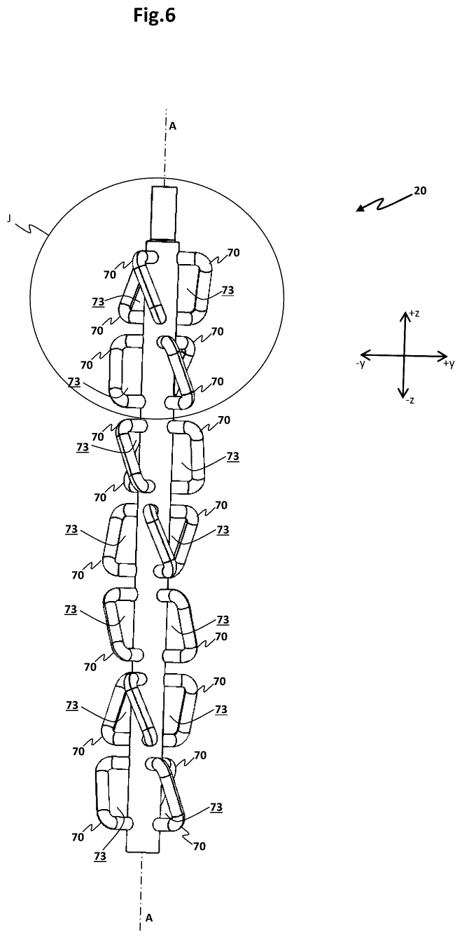

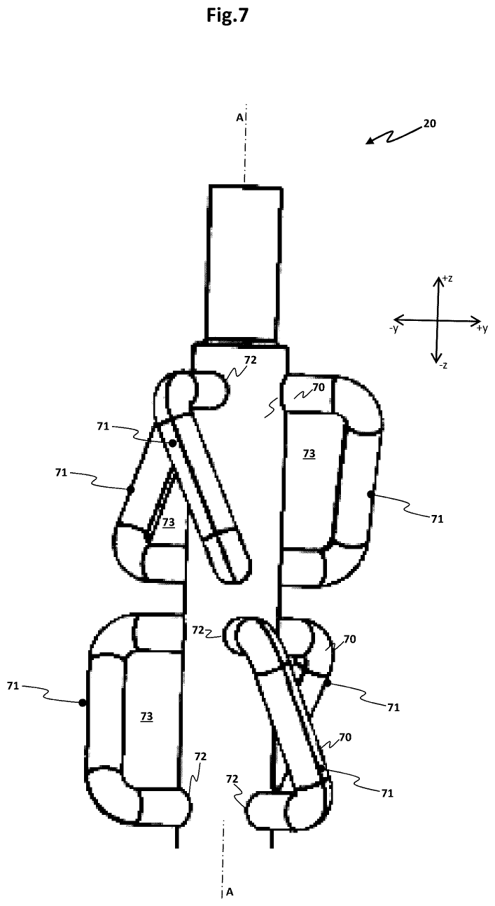

In an embodiment of any of the above-discussed versions of the proposed apparatus (1), the one or more protrusions (70) can be fixedly arranged to the respective shaft (20) over two or more junctions (72), thereby providing one or more holes (73) defined between the protrusion (70) and the shaft (20). This allows the passage of the waste through such holes (73), increases the shear exerted onto the waste, and provides an enhanced extent of mixing, thereby enhancing the homogeneity in processing of the waste. FIG. 6 shows an example in accordance with such embodiment, in which the one or more protrusions (70) are fixedly arranged to a shaft (20) over two junctions (72); thereby providing a handle-like shape to the protrusions (70) with a respective hole (73). FIG. 7 shows a close-up view of the detail J from FIG. 6. With reference to FIG. 7; the junctions (72) between a protrusion (70) and respective shaft (20) can be distributed at different alignments along the axis (A). That is, a first junction (72) can be at a first radial projection on the axis (A), and a further junction (72) can be at a second radial projection that is different from said first radial projection. Accordingly, such protrusion (70) can be considered to have a handle-shaped or C-shaped structure that is connected to the shaft at different loci along the axis (A). In any embodiment according to the present invention, the production of shafts (20) is easy and at a low-cost. Considering that the shafts (20) can be made of metallic material such as steel, the protrusions (70) can be formed merely by attaching the same onto the shaft (20), e.g., by welding at junction(s).

Further referring to FIG. 7; the junctions (72) between a protrusion (70) and respective shaft (20) can be distributed at different angular alignments along the axis (A). That is, for a protrusion (70), one of the junctions (72) can be provided at a first radial position around the axis (A), and another one of the junctions (72) can be provided at a second radial position around the axis (A); wherein the first radial position is different from the second radial position. As a result, such protrusion (70) has an oblique arrangement with regard to the axis (A). Considering that a cooperating protrusion (70) on an adjacently arranged further shaft (20); a local guiding force component parallel to the axis (A) (that is, oblique to the flow direction (FD)) is exerted to the waste stream. This enhances the mixing of the waste stream; and also enhances the kneading effect and grinding.

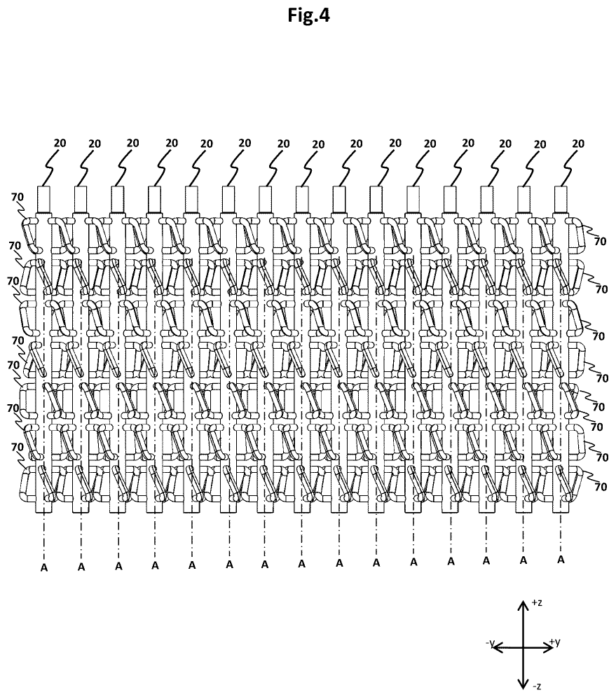

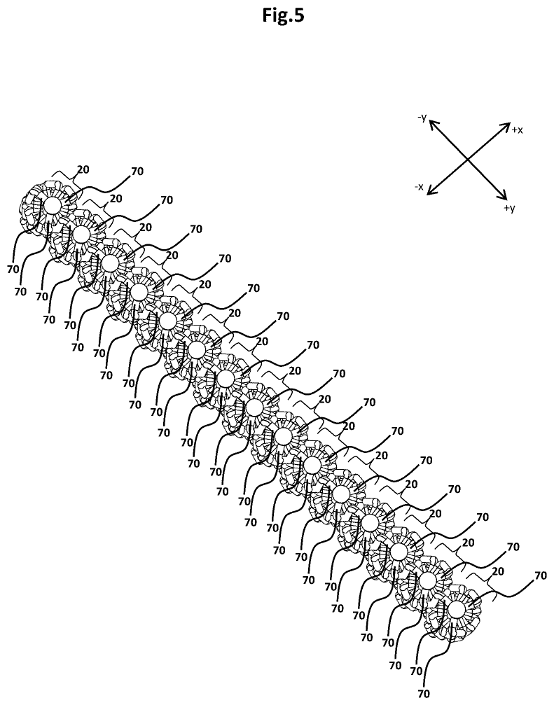

In a possible embodiment, a shaft (20) can comprise a plurality of protrusions (70) that are provided at an axial alignment in common and that are distributed in different radial directions around the axis (A). In other words, a plurality of protrusions (70) on a shaft (20) can be axially aligned with each other to extend in different radial directions with regard to the axis (A). Such shaft (20) effectively serves for grinding notwithstanding the momentary rotational position thereof. The number of protrusions (70) can be also thereby multiplied, thus enhancing the grinding and processing efficiency of the apparatus (1). Such arrangement of the protrusions (70) is exemplified in FIG. 2 to FIG. 7.

In an embodiment, one or more couples of shafts (20) that are arranged adjacent to each other can be provided with respective protrusions (70) that are at an overlapping axial alignment with each other.

More preferably, each shaft (20) in said couple of shafts (20) can be provided with a plurality of protrusions (70) at an axial alignment and equiangularly distributed around the axis (A). This provides a more stabilized rotation of the shaft thanks to balanced weight distribution of the protrusions (70) around the axis (A). For instance, at a group of three protrusions (70) that are radially distributed around the axis (A) at an axial locus in common, adjacently arranged protrusions (70) can have a radial angle of about 120° in-between each other. In other words, each shaft (20) in such couple of shafts (20) can be provided with three protrusions (70) at an axial alignment, that are distributed around the axis with an angle of 120 degrees in-between adjacently arranged protrusions (70).

Preferably, adjacently disposed shafts (20) can be provided with cooperating sets of obliquely arranged and radially distributed plurality of protrusions (70) at one or more axial alignments in common with each other. For instance, each protrusion (70) can comprise a first end and a second end that is at a different axial alignment relative to the axial alignment of the first end; and in a couple of shafts (20) that are adjacent to each other, respective protrusions (70) that have overlapping axial alignments are arranged such that, when in a stationary position, a distance between respective first ends is different from a distance between respective second ends. FIG. 2 to FIG. 4 depict exemplary versions according to such embodiment.

Particularly for the case where the waste is a high solid load cellulosic waste, when a distance between cooperating respective protrusions (70) of the two or more shaft(s) orthogonal to a respective axis (A) is lower than 0.7 cm, difficulties start arising in introduction of waste in-between a side surface (71) and corresponding complementary surface. On the other hand, when said distance is higher than 4 cm, shear starts to be insufficient to achieve an effective grinding of the waste. Therefore, it is preferred that said distance is within the range between 0.7 cm and 4 cm when the cooperating protrusions (70) are at respective angular positions to face each other. It is observed that, in terms of increased effectiveness in hydrolysis of the high solid load cellulosic wastes, an optimum zone between facilitated introduction of waste in-between a side surface (71) and corresponding complementary surface and achieving an effective shear, is available when said distance is within the range between 1.5 cm and 3 cm.

It is further observed that, in the case where the protrusions (70) are provided with indentations that have sharp edges or sharp angles (e.g., higher than 5° over a full angle of 360°), cellulose adheres into the indentations and fill the same. This decreases the efficiency in grinding of cellulose. Therefore, it is preferred that any indentations on the protrusions (70) have an angle that is up to 5°. For instance, the indentations can be rounded, curved, smoothened or wave-formed for avoiding sharp angles on the protrusions (70).

Within the above-indicated contexts, the term “corresponding complementary surface(s)” corresponds to a respective grinding zone that is closest to a respective side surface (71) of a protrusion (70) in terms of distance defined above.

Within the context of the present application, a row of shafts can be considered to be aligned along a lateral orientation (−y/+y) that is orthogonal to the flow direction (FD) and also to the gravity (g) when the apparatus (1) is in use.

Any of the embodiment of the apparatus (1) can comprise a plurality (e.g., two) of rows that are arranged along the flow direction (FD), thereby enabling a contact between an upstream-side row and the waste that is already contacted by a downstream-side row with regard to said upstream-side row.

Considering that an embodiment of such apparatus (1) can comprise a plurality (e.g., two) of rows that are arranged along the flow direction (FD), thereby enabling a contact between an upstream-side row and the waste that is already contacted by a downstream-side row with regard to said upstream-side row:
Preferably, the axes (A) of shafts (20) in different rows are arranged to be offset with regard to the flow direction (FD). This feature enables the grinding of the waste that passes through the gap(s) in the downstream-side row, by a subsequent row.

With reference to FIG. 1 to FIG. 3, the two or more shaft(s) (20) can be provided on a carrier (30) to arrange that said shaft(s) (20) are translationally stationary yet rotationally movable relative to each other.

In a preferred embodiment of any of the above-discussed versions of the proposed apparatus (1), the apparatus (1) comprises a carrier (30) that is arranged to provide a first translational movement to the two or more shaft(s) (20) relative to the channel (10) along the flow direction (FD). Referring to the appending drawings; within the context of the present invention, the flow direction (FD) corresponds to a downstream direction (+x) on a −x/+x orientation. So, alternating components of the reciprocations in first translational movement can be considered to be a movement in upstream direction (+x), and another movement in downstream direction (−x).

Translating the shafts (20) in the upstream direction (−x) guarantees that plurality of portions of the waste are ground only once at a grinding round. Thus, the uniformity of hydrolysis is enhanced throughout the channel (10).

Accordingly, a preferred version of the method includes provision of a first translational movement to the two or more shaft(s) (20) relative to the channel (10) along the flow direction (FD). With these corresponding measures attributed to the apparatus (1) and method according to the present invention, the two or more shaft(s) (20) can be guided to exert the grinding along the channel (10). Thus, the uniformity in processing of the waste is enhanced. The first translational movement is to be considered as a first reciprocation that includes the following:
i. a translation of the two or more shaft(s) (20) along the channel (10) in the flow direction (FW) (that is, in downstream direction (+x)) (e.g., whilst grinding of a respective waste), and then
ii. a transfer of said two or more shaft(s) (20) back in an upstream direction (−x) to iterate said translation (e.g., a returning state for preparing to repeat the grinding state).

FIG. 1 represents an exemplary apparatus (1) according to the present embodiment, and shows that the two or more shaft(s) (20) are at an upstream end of the channel (10), and ready to be translated along the channel (10) in the flow direction (FW).

In a preferred embodiment, the carrier (30) is arranged to provide that the first translational movement of the two or more shaft(s) (20) is orthogonal to their respective axes (A). This feature enables the effective use of a smallest possible height in shaft(s) (20) along respective axes (A).

In a preferred embodiment of any of the above-discussed versions of the proposed apparatus (1), the carrier (30) is arranged to provide a second translational movement to the two or more shaft(s) (20) relative to the channel (10) orthogonal to the flow direction (FD). Accordingly, a preferred version of the method includes provision of a second translational movement to the two or more shaft(s) (20) relative to the channel (10) orthogonal to the flow direction (FD). With these corresponding measures attributed to the apparatus (1) and method according to the present invention, the two or more shaft(s) (20) can be kept away from the stream at transferring back in the upstream direction, thereby avoiding any unnecessary frictional losses and any obstruction of the waste stream flow in the flow direction (FD).

Referring to the appended drawings; within the context of the present invention, said second translational movement takes place in an −z/+z orientation. The −z/+z orientation is orthogonal to the flow direction (FD) that is on the −x/+x orientation. So, when the apparatus (1) is in use, alternating components of the reciprocations in second translational movement can be considered to be a movement in an upwards direction (+z), and another movement in a downwards direction (−z).

Considering that a waste stream is supported by a bottom of the channel (10) against gravity (g); the second translational movement is to be considered as a second reciprocation that includes the following:
a) a temporary extraction of the shaft(s) (20) from the channel, thus a temporary separation of the shaft(s) (20) from the stream, by moving the shaft(s) (20) in the upwards direction (+z) (that is, away from the bottom of the channel (10)); and then
b) a (re-)introduction of the shaft(s) (20) into the channel, by moving the shaft(s) (20) in the downwards direction (−z) (that is, towards the bottom of the channel (10)), thus into the stream.

Within this context, FIG. 1 exemplifies a positioning at a state (first position) where the two or more shaft(s) (20) are introduced (or re-introduced) in the channel (10), for instance, by being extended from the carrier (30). Here, the shafts (20) are in a state where they are translated (here: introduced, extended or dipped) into the channel, in a direction orthogonal to the flow direction (FD), for instance, in accordance with the gravity (g). Thus, in such state, contact between the two or more shaft(s) (20) and a respective waste stream is enabled.

The shaft(s) (20) can be retracted by the carrier (30) to be positioned (here: elevated) for separation/extraction from a waste stream flowing through the channel (10). Such extraction can be performed, for instance, by retracting the shaft(s) (20) into the carrier (30). Such positioning can be achieved for instance prior to commencement of a hydrolysis procedure, or at cleaning of the apparatus (1), or at an instance where the shaft(s) (20) are just returned from a downstream end of the channel (10) to be reintroduced into the waste stream at an upstream end of the channel (10).

The elevation of the shafts (20) by the carrier (30) enables the avoidance from contact between the shaft(s) (20) and a respective waste stream, thereby facilitating the first translational movement of the shaft(s) (20) along the flow direction (FD) relative to the channel (10).

Exemplary detailed information on the process using the apparatus (1) according to the present application:
1) The shaft(s) (20) can be rotated at a very low rate (here: rotational speed): for instance, up to 10 rpm; The apparatus (1) can be provided with one or more reduction gears (not shown) for determination/modulation of rotational speed of said two or more shafts (20). Thus, a high extent of torque can be achieved along with low rotational rate. As a result, the mechanical energy consumed by the apparatus (1) can be substantially attributed to grinding.
2) The shaft(s) (20) can be translated along the flow direction (FD) at a very low speed: for instance, up to 50 cm/min.
3) It can be contemplated that;
   when the shafts (20) are protruded into the channel, the first translational movement in the downstream direction (+x) corresponds to promoting the waste in the flow direction (FW);
   a general linear velocity of the waste stream flowing through the channel (10) can be determined via manipulating the speed of first translational movement; thus, the residence time can be manipulated.
4) When the shafts (20) reach to a downstream end of the channel as a result of the first translational movement in the downstream direction (+x), the carrier (30) can be operated to separate the shafts (20) from the channel (10) by moving them away from the base in the upwards direction (+z). Then, the carrier (30) can be further operated to translate the shafts (20) in the upstream direction (−x). As a result, the shafts (20) can be brought to e.g., an upstream end of the channel (10), and then re-introduced into the channel (10) by moving/translating them in the downwards direction (−z).
5) The shafts (20) can be translated again as in the item (2) above, corresponding to an iteration of the grinding of the waste and promoting of the same along the flow direction (FD). In the case where non-hydrolysed waste is fed from the upstream end and a corresponding amount of (at least partially-) hydrolysed waste is simultaneously streamed away (that is, taken or removed) from the downstream end, the apparatus (1) corresponds to a continuous hydrolysis reactor; (in another approach, the hydrolysed waste can be re-introduced into the channel (10) through the upstream end, and in such case, the apparatus (1) corresponds to a re-circulating or semi-batch hydrolysis reactor).
6) Within a pre-determined residence time, the iteration can be repeated (e.g., 100 to 200 times) to exert grinding onto the waste when streaming through the channel (10). With an increased number of rows, a rate of grinding can be increased even with a reduced number of repetition or iteration.
7) The solid load of the cellulosic waste provides a high extent of internal friction coefficient to the waste, and reduces the fluidity of the same. So, effectiveness of grinding is increased with such high solid load. Within the context of the present application, for cellulosic wastes such as paper sludge are considered to have a high solid load when the solid load is within the range between 35 % (wt.) and 40 % (wt.) with regard to the total weight of said waste.
8) As an indicator of extent of hydrolysis, viscosity refers to corresponding extent of decomposition of macromolecular cellulose structure at the end of the residence time. Accordingly, as the hydrolysis proceeds in the channel (10), the viscosity of the waste gradually decreases, resulting in a gradual decrease in effectiveness of grinding. Hence, the effectiveness in grinding (also the contribution for an effective hydrolysis) can be considered to have a maximum at the earlier stages of the operation of the apparatus (1), and gradually decreases throughout the residence time. The grinding is considered to be neither effective nor necessary at late stages of hydrolysis in which the cellulose is almost completely converted into glucose, and in which the viscosity reaches to a possible minimum value. Accordingly, the apparatus (1) and method according to the present invention can be considered to be most useful in a “partial hydrolysis” of high solid load cellulosic wastes, in particular, of wastes that contain crystalline cellulose. As an example to partial hydrolysis: a waste stream can have a mean glucose number of 10000 at entering the apparatus (1), and can be hydrolysed throughout the residence time until the mean glucose monomer number of cellulose molecules is decreased to a range between 5 and 5000. In other words, the hydrolysis can be performed in a partial extent such that, at exiting the apparatus (1), the waste stream can have a mean glucose monomer number of cellulose molecules at a range between 5 and 5000, preferably of up to 1000, for instance 100.
9) For effecting the hydrolysis during the residence time, the respective method can be considered to include the introduction of one or more enzymes (cellulases) or one or more cellulolytic microorganisms/bacteria (e.g., Cellulomonas fimi or Bacillus amyloliquefaciens) into the waste to be processed in the apparatus (1).
10) The apparatus (1) can include one or more heating means and one or more temperature sensor(s), preferably in communication with a temperature controller, in order to adjust waste temperature at an optimal level for hydrolysis.
11) Along with the grinding function, the protrusions can be considered to constitute a mixing means that contributes in enhancing the uniformity of the waste in the channel (10).

### REFERENCE SIGNS

1 apparatus
10 channel
20 shaft
30 carrier
70 protrusion
71 side surface
72 junction
73 hole
A axis
FD flow direction
g gravity
+X downstream direction
−x upstream direction
−y/+y lateral orientation
+Z upwards direction
−Z downwards direction

1. An apparatus (1) for use in cellulose hydrolysis, comprising:
a channel (10) for guiding a stream of cellulose-containing waste in a flow direction (FD);
two or more shaft(s) (20) arranged to rotate around respective axes (A) that are parallel to each other; wherein
the two or more shafts (20) include one or more protrusions (70) that radially extend away from the axis (A).

2. The apparatus according to claim 1, wherein side surfaces (71) of protrusions (70) are provided with indentations.

3. The apparatus according to claim 1, wherein the two or more shaft(s) (20) are arranged to have their respective axes (A) extending along the gravity vector (g) and orthogonal to the flow direction (FD) when the apparatus (1) is in use.

4. The apparatus according to claim 1, wherein said two or more shaft(s) (20) are each arranged to rotate in a respective rotational direction that is opposite to the one or more adjacently arranged shaft(s).

5. The apparatus according to claim 1, wherein said two or more shaft(s) (20) are each arranged to rotate such that respective side surfaces (71) on the protrusions (70) exhibit a relative linear velocity higher than zero with respect to complementary side surfaces (71) on protrusions (70) of adjacently arranged shaft(s) (20).

6. The apparatus according to claim 1, wherein the one or more protrusions (70) are fixedly arranged to the respective shaft (20) over two or more junctions.

7. The apparatus according to claim 6, wherein the junctions between a protrusion (70) and respective shaft (20) are distributed at different alignments along the axis (A).

8. The apparatus according to claim 1, wherein the shaft (20) comprises a plurality of protrusions (70) that are provided at an axial alignment in common and that are distributed in different radial directions around the axis (A).

9. The apparatus according to claim 1, wherein one or more couples of shafts (20) that are arranged adjacent to each other are provided with respective protrusions (70) that are at an overlapping axial alignment with each other.

10. The apparatus according to claim 9, wherein each shaft (20) in said couple of shafts (20) is provided with a plurality of protrusions (70) at an axial alignment, said plurality of protrusions (70) being equiangularly distributed around the axis (A).

11. The apparatus according to claim 10, wherein each shaft (20) in said couple of shafts (20) is provided with three protrusions (70) at an axial alignment, that are distributed around the axis with an angle of 120 degrees in-between adjacently arranged protrusions (70).

12. The apparatus according to claim 1, wherein one or more of the protrusions (70) are obliquely arranged with regard to the axis (A).

13. The apparatus according to claim 12, wherein each protrusion (70) comprises a first end and a second end that is at a different axial alignment relative to the axial alignment of the first end; and in a couple of shafts (20) that are adjacent to each other, respective protrusions (70) that have overlapping axial alignments are arranged such that, when in a stationary position, a distance between respective first ends is different from a distance between respective second ends.

14. The apparatus according to claim 1, comprising a carrier (30) arranged to provide a first translational movement to the one or more shaft(s) (20) relative to the channel (10) along the flow direction (FD).

15. The apparatus according to claim 14, wherein the carrier (30) is arranged to provide that the first translational movement of the one or more shaft(s) (20) is orthogonal to their respective axes (A).

16. The apparatus according to claim 14, wherein the carrier (30) is arranged to provide a second translational movement to the one or more shaft(s) (20) along an orientation of gravity (g) when the apparatus (1) is in use.

17. A method for cellulose hydrolysis, including the following actions:
guiding a stream of cellulose-containing waste in a flow direction (FD) along a channel (10); and
grinding the waste between respective protrusions (70) that radially extend away from respective axes (A) of two or more shafts (20) that rotate around an axis (A).

18. The method according to claim 17, further including provision of a first translational movement to the one or more shaft(s) (20) relative to the channel (10) along the flow direction (FD).

19. The method according to claim 17, further including provision of a second translational movement to the one or more shaft(s) (20) relative to the channel (10) orthogonal to the flow direction (FD).
